SEQUENCE LISTING (1 sequence)

SEQ ID NO: 1 - protein, 687 aa, Homo sapiens
MAEELVLERCDLELETNGRDHHTADLCREKLVVRRGQPFWLTLHFEGRNYEASVDSLTFSVVTGPAPSQEAGTKARFPLRDAVEEGDWTATVVDQQDCTLSLQLTTPANAPIGLYRLSLEASTGYQGSSFVLGHFILLFNAWCPADAVYLDSEEERQEYVLTQQGFIYQGSAKFIKNIPWNFGQFEDGILDICLILLDVNPKFLKNAGRDCSRRSSPVYVGRVVSGMVNCNDDQGVLLGRWDNNYGDGVSPMSWIGSVDILRRWKNHGCQRVKYGQCWVFAAVACTVLRCLGIPTRVVTNYNSAHDQNSNLLIEYFRNEFGEIQGDKSEMIWNFHCWVESWMTRPDLQPGYEGWQALDPTPQEKSEGTYCCGPVPVRAIKEGDLSTKYDAPFVFAEVNADVVDWIQQDDGSVHKSINRSLIVGLKISTKSVGRDEREDITHTYKYPEGSSEEREAFTRANHLNKLAEKEETGMAMRIRVGQSMNMGSDFDVFAHITNNTAEEYVCRLLLCARTVSYNGILGPECGTKYLLNLNLEPFSEKSVPLCILYEKYRDCLTESNLIKVRALLVEPVINSYLLAERDLYLENPEIKIRILGEPKQKRKLVAEVSLQNPLPVALEGCTFTVEGAGLTEEQKTVEIPDPVEAGEEVKVRMDLLPLHMGLHKLVVNFESDKLKAVKGFRNVIIGPA

(19) United States
(12) Patent Application Publication
(10) Pub. No.: US 2023/0414700 A1
(43) Pub. Date: Dec. 28, 2023

(54) TG2 INHIBITORS FOR IMPROVING MUCOCILIARY CLEARANCE IN RESPIRATORY DISEASES

(71) Applicants: INSERM (INSTITUT NATIONAL DE LA SANTÉ ET DE LA RECHERCHE MÉDICALE), Paris (FR); UNIVERSITÉ DE PARIS, Paris (FR); SORBONNE UNIVERSITÉ, Paris (FR); UNIVERSITÉ DE LILLE, Lille (FR); ASSISTANCE PUBLIQUE-HÔPITAUX DE PARIS (APHP), Paris (FR)

(72) Inventors: Séverine LETUVE, Paris (FR); Céline SALLON, Paris (FR); Loïc GUILLOT, Paris Cedex 12 (FR); Catherine ROBBE MASSELOT, Villeneuve d Ascq (FR); Camille TAILLE, Paris (FR)

(21) Appl. No.: 18/031,251

(22) PCT Filed: Oct. 14, 2021
(86) PCT No.: PCT/EP2021/078534

§ 371 Date: Apr. 11, 2023

(30) Foreign Application Priority Data
Oct. 15, 2020 (EP) 20306214.6

(51) Int. Cl. A61K 38/07; A61K 45/06; A61P 11/12

(52) U.S. Cl. CPC A61K 38/07; A61P 11/12; A61K 45/06

(57) ABSTRACT

In asthma, modification of gel-forming respiratory mucins leading to their tethering to the apical pole of epithelial cells, are believed to participate in airway obstruction by mucus plugs. These changes have been linked to local production of Th2 cytokines, resulting in mucus cell hyperplasia and increased MUC5AC production. The inventors showed that severe eosinophil asthma was associated with overexpression of transglutaminase 2 (TG2), an enzyme recently involved in intestinal mucin reticulation. Moreover, the bronchial epithelium from asthmatic patients or control subjects was reconstituted in vitro by culturing cells at the air-liquid interface and the hypersecretory differentiation was modeled by exposing control bronchial epithelial to IL-13. The inventors showed TG2 expression was upregulated upon IL-13-mediated hypersecretory differentiation and correlated with MUC5AC expression. IL-13 promoted MU5AC tethering to in vitro reconstituted hypersecretory epithelium, and this was blocked by a TG2 inhibitor. In conclusion, the inventors showed that TG2 participates in respiratory mucin modifications in asthma, and contribute to mucus tethering to the airway wall, supporting the use of TG2 inhibitors for improving mucociliary clearance in asthma, but more generally in respiratory diseases.

### FIELD OF THE INVENTION

The present invention is in the field of medicine, especially in respiratory diseases.

### BACKGROUND OF THE INVENTION

Mucus represents the first line of defense of our respiratory tract. Mucociliary clearance is essential for maintaining the homeostasis of airway epithelium and for anti viral/anti bacterial defenses. When it becomes dysfunctional, the hypersecretion and accumulation of mucus, associated with changes in its biophysical properties, can become the major contributor to chronic airway diseases such as CPOD, cystic fibrosis or asthma. For instance, mucus plugging of airways can represent the main cause of death during an acute severe asthma exacerbation. However, there is a lack for effective therapies that could be suitable for decreasing the viscoelasticity of mucus and its adhesiveness to the respiratory epithelium, and thus accelerating the rate of mucociliary clearance.

Transglutaminase 2 (EC 2.3.2.13; TG2 or TGase 2) plays important roles in the pathogenesis of many diseases, including inflammatory disorders. Accordingly, TG2 is considered to be a potential therapeutic target for said diseases. Many data state that TG2 inhibitors would be suitable for the treatment of asthma but only in perspective to damper the inflammation process. The role of TG2 in the mucociliary clearance has never been yet investigated.

### SUMMARY OF THE INVENTION

The present invention is defined by the claims. In particular, the present invention relates to use of TG2 inhibitors for improving mucociliary clearance in chronic airway diseases.

### DETAILED DESCRIPTION OF THE INVENTION

The first object of the present invention relates to a method of improving mucociliary clearance in a patient suffering from a chronic airway disease, comprising administration of a therapeutically effective amount of TG2 inhibitor.

The present invention also relates to a method of increasing the liquefaction of mucus in a patient suffering from a chronic airway disease, comprising administering to the subject a therapeutically effective amount of TG2 inhibitor.

The present invention also relates to a method of decreasing the viscoelasticity of airway mucus in a patient suffering from a chronic airway disease comprising administering to the subject a therapeutically effective amount of TG2 inhibitor.

The present invention also relates to a method of preventing mucus tethering to the airway wall in a patient suffering from chronic airway disease comprising administering to the subject a therapeutically effective amount of TG2 inhibitor.

The present invention also relates to a method of preventing extensive airway mucus plugging in a patient suffering from chronic airway disease comprising administering to the subject a therapeutically effective amount of TG2 inhibitor.

The present invention also relates to a method of preventing mucus occlusion of an airway lumen comprising administering to the subject a therapeutically effective amount of TG2 inhibitor.

As used herein, the term “patient” is interchangeable with the term “individual” or “subject”, and may refer to a subject to be treated by the methods disclosed herein. In some embodiments, the patient is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In some embodiments, the mammal is a human.

According to the present invention the respiratory disease thus features aberrant mucus production. In particular, the patient has excessively viscous or adhesive mucus. Thus in some embodiments, the patient has a chronic airway disease in which abnormal or excessive viscoelasticity or cohesiveness of mucus. Examples of diseases that may, at least some of the time, be associated with abnormal or excessive viscoelasticity and/or adhesiveness of the mucus, and when such a symptom occurs, include, but are not limited to cystic fibrosis; chronic or acute bronchitis; bronchiectasis (non-CF and CF bronchiectasis); acute tracheitis (bacterial, viral, mycoplasmal or caused by other organisms); acute or chronic sinusitis; atelectasis (lung or lobar collapse) resulting from acute or chronic mucus plugging of the airways (sometimes seen in a variety of diseases such as asthma); and bronchiolitis (viral or other). In addition, the methods of the present invention may be useful for reducing symptoms associated with excessive viscosity and/or cohesiveness of the mucus in patients with a variety of respiratory infections, including both viral and bacterial infections.

In some embodiments, the patient has a chronic airway disease selected from, cystic fibrosis (CF), chronic obstructive pulmonary disease, bronchiectasis and asthma.

In some embodiments, the patient suffers from asthma and in particular severe asthma.

As used herein, the term “asthma” refers to diseases that present as reversible airflow obstruction and/or bronchial hyper-responsiveness that may or may not be associated with underlying inflammation. Examples of asthma include allergic asthma, atopic asthma, corticosteroid naive asthma, chronic asthma, corticosteroid resistant asthma, corticosteroid refractory asthma, asthma due to smoking, asthma uncontrolled on corticosteroids and other asthmas as mentioned, e.g., in the Expert Panel Report 3: Guidelines for the Diagnosis and Management of Asthma, National Asthma Education and Prevention Program (2007) (“NAEPP Guidelines”), incorporated herein by reference in its entirety.

As used herein, the term “severe asthma” has its general meaning in the art and refers to asthma which requires treatment with high doses of corticosteroid and β₂-adrenergic receptor agonist to prevent it from becoming uncontrolled or which remains uncontrolled despite therapy.

As used herein, the term “COPD” as used herein refers to chronic obstructive pulmonary disease. The term “COPD” includes two main conditions: emphysema and chronic obstructive bronchitis.

As used herein the term “cystic fibrosis” has its general meaning in the art and refers to an inherited autosomal disease associated with mutations to the gene encoding the cystic fibrosis transmembrane conductor regulator (CFTR). The method of the invention may be performed for any type of cystic fibrosis such as revised in the World Health Organisation Classification of cystic fibrosis and selected from the E84 group: mucoviscidosis, Cystic fibrosis with pulmonary manifestations, Cystic fibrosis with intestinal manifestations and Cystic fibrosis with other manifestations. In some embodiments, the subject harbours at least one mutation in the CFTR gene, including, but not limited to F508del-CFTR, R117H CFTR, and G551D CF TR (see, e.g., http://www.genet.sickkids.on.ca/cftr, for CF TR mutations).

As used herein, the term “mucus” has its general meaning in the art and refers to a usually clear viscid fluid that is secreted by mucous membranes and glandes of the respiratory tract. Mucus moistens, lubricates and protects the tissues from which it is secreted. It comprises mucin macromolecules, which are the gel forming constituents of mucus. The viscoelastic properties of normal mucus are dependent on the concentration, molecular weight, and entanglements between mucin polymers.

As used herein, the term “mucociliary clearance” refers to the ability of the mucus to be cleared from the respiratory tract of the patient. Thus the expression “improving mucociliary clearance” is any improvement of mucus clearance from a starting level. This would be determined by the ability of a patient to eject mucus from the respiratory tract. The terms would be understood by a person of ordinary skill in the art.

As used herein the term “liquefaction” refers to the act of becoming liquid. Therefore, an increase in the liquefaction of mucus refers to the increase in liquid phase or liquid state of mucus, as compared to a more solid or viscous phase. The method of the present invention is thus used to increase liquefaction of the mucus or sputum and provide at least some relief or therapeutic benefit to the patient.

As used herein, the expression “decreasing the viscoelasticity of mucus” refers to a decrease in viscoelasticity as measured by known methods as compared to a starting or initial measurement of viscoelasticity of the respiratory tract mucus of the patient. Similarly with the expression improving mucociliary clearance, the expression “decreasing the viscoelasticity of mucus” means that the mucus clearance is accelerated by a decrease in mucus viscoelasticity.

As used herein, the term “mucus tethering to the airway wall” refers to the ability of the mucus to be adhered to the respiratory epithelium. Therefore, the expression “preventing mucus tethering to the airway wall” is any decrease in mucus attached to the airway surface or any increase in mucus release from the airway epithelium.

As used herein, the term “extensive airway mucus plugging” refers to a large number of occluded airways caused by mucus plugs (e.g. completely occluded airways) in one or more segments of the lungs. Identification of extensive airway mucus plugging may be determined by assessing the quantity of mucus in an airway within the lung of the subject. In some embodiments, extensive airway mucus plugging can indicate complete occlusion of about 5-10%, about 10-20%, about 20-30%, about 30-40%, about 40-50%, about 50-60%, about 60-70%, about 70-80%, about 80-90%, about 90-100%, or about 5%, 10%, 15%, 20%, 30%, 40%, 50%, 60%, 70%, 80%, 90%, or 95% of segments.

As used herein, the term “mucus occlusion of an airway lumen” indicates a complete opacification of an airway by mucus with or without bronchial dilatation as indicated by, e.g., lung imaging. In embodiments, mucus plugs can be detected (e.g. seen) in sections (such as longitudinal sections) as tubular structures with or without branching or in cross-section as rounded opacities.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein the term “Transglutaminase 2” or “TG2” has its general meaning in the art and refers to the tissue transglutaminase, abbreviated tTG or TG2. An exemplary amino acid sequence for TG2 is shown as SEQ ID NO:1.

>sp|P21980|TGM2_HUMAN Protein-glutamine gamma-
glutamyltransferase 2 OS = Homo sapiens OX = 9606
GN = TGM2 PE = 1 SV = 2
SEQ ID NO: 1
MAEELVLERCDLELETNGRDHHTADLCREKLVVRRGQPFWLTLHFEGRN
YEASVDSLTESVVTGPAPSQEAGTKARFPLRDAVEEGDWTATVVDQQDC
TLSLQLTTPANAPIGLYRLSLEASTGYQGSSFVLGHFILLENAWCPADA
VYLDSEEERQEYVLTQQGFIYQGSAKFIKNIPWNFGQFEDGILDICLIL
LDVNPKFLKNAGRDCSRRSSPVYVGRVVSGMVNCNDDQGVLLGRWDNNY
GDGVSPMSWIGSVDILRRWKNHGCQRVKYGQCWVFAAVACTVLRCLGIP
TRVVTNYNSAHDQNSNLLIEYFRNEFGEIQGDKSEMIWNFHCWVESWMT
RPDLQPGYEGWQALDPT

PQEKSEGTYCCGPVPVRAIKEGDLSTKYDAPFVFAEVNADVVDWIQQDD
GSVHKSINRSLIVGLKISTKSVGRDEREDITHTYKYPEGSSEEREAFTR
ANHLNKLAEKEETGMAMRIRVGQSMNMGSDFDVFAHITNNTAEEYVCRL
LLCARTVSYNGILGPECGTKYLLNLNLEPFSEKSVPLCILYEKYRDCLT
ESNLIKVRALLVEPVINSYLLAERDLYLENPEIKIRILGEPKQKRKLVA
EVSLQNPLPVALEGCTFTVEGAGLTEEQKTVEIPDPVEAGEEVKVRMDL
LPLHMGLHKLVVNFESDKLKAVKGERNVIIGPA

As used herein, the term “TG2 inhibitor” should be understood broadly and encompasses any substance able to prevent the action of TG2, and encompasses inhibitors of TG2 activity and inhibitors of TG2 expression. The term “inhibitor of TG2 activity” should be understood broadly and encompasses substances acting directly on TG2 and able to prevent the interaction or binding between TG2 and its ligands. So, said inhibitor of TG2 activity particularly encompasses classical inhibitors of TG2 activity which are small organic molecules well known in the art. In some embodiments, the TG2 inhibitor is an inhibitor of TG2 gene expression. An “inhibitor of gene expression” refers to a natural or synthetic compound that has a biological effect to inhibit or significantly reduce the expression of a gene. Consequently an “inhibitor of TG2 gene expression” refers to a natural or synthetic compound that has a biological effect to inhibit or significantly reduce the expression of the gene encoding the TG2 protein.

In some embodiments, the TG2 inhibitor be selected from the group of competitive amine inhibitors (Lorand et al, 1984). Some of the most commonly used competitive amine inhibitors, including putrescine, monodansylcadaverine (MDC), 5-(biotinamido) pentylamine, cystamine, spermidine, histamine and fluorescin cadaverine.

In some embodiments, the TG2 inhibitor is selected from the group of reversible TG2 inhibitors. Examples of reversible TG2 inhibitors are GTP and GDP, the divalent metal ion Zn2+ or GTP analogues such as GTPγS and GMP-PCP (Lai et al., 1998; Lorand et al., 1984; Aeschlimann et al., 1994).

In some embodiments, the TG2 inhibitors is selected from the group of irreversible TG2 inhibitors such as iodoacetamide (Folk et al., 1966; de Macedo et al., 2000), 3-halo-4,5-dihydroisoxazoles compounds, gluten peptides or 2-[(2-oxopropyl)thio]imidazolium derivatives (Freund et al., 1994; Hausch et al., 2003, Maiuri et al., 2005), or Cbz-gln-gly analogues derived from TG2 substrate carbobenzyloxy-Lglutaminylglycine (Cbz-gln-gly) as the inhibitor backbone (de Macedo et al., 2002; Pardin et al., 2006).

In some embodiments, the TG2 inhibitor is selected from peptidomimetic irreversible inhibitors using a gluten peptide sequence as the inhibitor backbone (Hausch et al., 2003).

In some embodiments, the TG2 inhibitors is selected from substituted 3,4-dihydrothieno [2,3-d] pyrimidines (WO 2006060702) such as thieno[2,3-d]pyrimidin-4-one acylhydrazide (Duval et al, 2005; Case et al, 2005) and from substituted cinnamoyl benzotriazolyl amides and the 3-(substituted cinnamoyl)pyridines (Pardin et al., 2008).

In some embodiments, the TG2 inhibitor is GK-921 or GK-428 having the formula of:

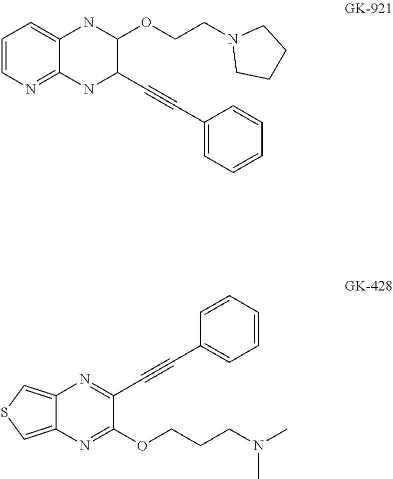

In some embodiments, the TG2 inhibitor is ZDON having the formula of:

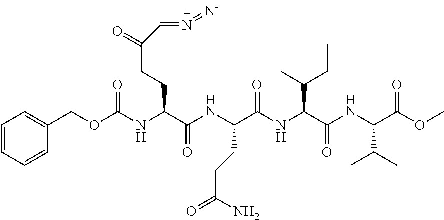

In some embodiments, the TG2 inhibitor is an antibody or antibody fragment that can partially or completely blocks the action of TG2 on its substrates (or the interaction between TG2 and its substrates). In particular, the TG2 inhibitor consists in an antibody directed against TG2, in such a way that said antibody blocks the binding of TG2 on its substrates. More particularly, said antibody can be directed against the active site of TG2.

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995).

Antibodies directed against TG2 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies of the invention can be prepared and isolated using any technique that provides for the production of antibody molecules by continuous cell lines in culture. Techniques for production and isolation include but are not limited to the hybridoma technique originally described by Kohler and Milstein; the human B-cell hybridoma technique and the EBV-hybridoma technique. Alternatively, techniques described for the production of single chain antibodies (see, e.g., U.S. Pat. No. 4,946,778) can be adapted to produce single chain antibodies of the invention. Inhibitors of TG2 activity useful in practicing the present invention also include antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity.

In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”.

In some embodiments, the antibody of the present invention is a scFv. As used herein, the term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

In some embodiments, the antibody is a humanized antibody. As used herein, the term “humanized” describes antibodies wherein some, most or all of the amino acids outside the CDR regions are replaced with corresponding amino acids derived from human immunoglobulin molecules.

Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. As used herein, the term “fully human” refers to an immunoglobulin, such as an antibody or antibody fragment, where the whole molecule is of human origin or consists of an amino acid sequence identical to a human form of the antibody or immunoglobulin. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, said inhibitor of gene expression is a siRNA, an antisense oligonucleotide or a ribozyme. For example, anti-sense oligonucleotides, including anti-sense RNA molecules and anti-sense DNA molecules, would act to directly block the translation of TG2 mRNA by binding thereto and thus preventing protein translation or increasing mRNA degradation, thus decreasing the level of TG2, and thus activity, in a cell. For example, antisense oligonucleotides of at least about 15 bases and complementary to unique regions of the mRNA transcript sequence encoding TG2 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. TG2 gene expression can be reduced by contacting a patient or cell with a small double stranded RNA (dsRNA), or a vector or construct causing the production of a small double stranded RNA, such that TG2 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing TG2. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

As used herein, the expression “therapeutically effective amount” is meant a sufficient amount of the TG2 inhibitor at a reasonable benefit/risk ratio applicable to any medical treatment. It will be understood that the total daily usage of the compound will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the TG2 inhibitor may be combined with pharmaceutically acceptable excipients, and optionally sustained-release matrices, such as biodegradable polymers, to form pharmaceutical compositions. The term “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The TG2 inhibitor can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The present invention also relates to a method for increasing the lung delivery of nanoparticles in a patient in need thereof comprising administering the nanoparticles in combination with an amount of a TG2 inhibitor as described herein.

Typically the subject suffers from a respiratory disease as described above.

As used herein, the term “nanoparticle” generally refers to a particle of any shape having a diameter from about 1 nm up to, but not including, about 1 micron, more preferably from about 5 nm to about 500 nm, most preferably from about 5 nm to about 100 nm. Nanoparticles having a spherical shape are generally referred to as “nanospheres”. For most nanoparticles, the size of the nanoparticles is the distance between the two most distant points in the nanoparticle. Nanoparticle size can be determined by different methods such as Dynamic Light Scattering (DLS), Small Angle X-ray Scattering (SAXS), Scanning Mobility Particle Sizer (SMPS), Scanning Electron Microscopy (SEM), Transmission Electron Microscopy (TEM) (Orts-Gil, G., K. Natte, et al. (2011), Journal of Nanoparticle Research 13(4): 1593-1604; Alexandridis, P. and B. Lindman (2000), Amphiphilic Block Copolymers: Self-Assembly and Applications, Elsevier Science; Hunter, R. J. and L. R. White (1987). Foundations of colloid science, Clarendon Press.).

Typically, the nanoparticles are made of biocompatible polymers. Any number of biocompatible polymers can be used to prepare the nanoparticles. In some embodiments, the biocompatible polymer(s) is biodegradable. In some embodiments, the particles are non-degradable. In some embodiments, the particles are a mixture of degradable and non-degradable particles.

As used herein, the term “biocompatible” or “biologically compatible” refer to materials that are, along with any metabolites or degradation products thereof, generally non-toxic to the recipient, and do not cause any significant adverse effects to the recipient. Generally speaking, biocompatible materials are materials which do not elicit a significant, inflammatory or immune response when administered to a patient.

Exemplary polymers include, but are not limited to, cyclodextrin-containing polymers, in particular cationic cyclodextrin-containing polymers, such as those described in U.S. Pat. No. 6,509,323; polymers prepared from lactones, such as poly(caprolactone) (PCL); polyhydroxy acids and copolymers thereof such as poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(D,L-lactide-co-caprolactone), poly(D,L-lactide-co-caprolactone-co-glycolide), poly(D,L-lactide-co-PEO-co-D,L-lactide), poly(L-lactide-co-PPO-co-D,L-lactide), and blends thereof, polyalkyl cyanoacrylate, polyurethanes, polyamino acids such as poly-L-lysine (PLL), poly(valeric acid), and poly-L-glutamic acid; hydroxypropyl methacrylate (HPMA); polyanhydrides; polyesters; polyorthoesters; poly(ester amides); polyamides; poly(ester ethers); polycarbonates; polyalkylenes such as polyethylene and polypropylene; polyalkylene glycols such as poly(ethylene glycol) (PEG) and polyalkylene oxides (PEO), and block copolymers thereof such as polyoxyalkylene oxide (“PLURONICS®”); polyalkylene terephthalates such as poly(ethylene terephthalate); ethylene vinyl acetate polymer (EVA); polyvinyl alcohols (PVA); polyvinyl ethers; polyvinyl esters such as poly(vinyl acetate); polyvinyl halides such as poly(vinyl chloride) (PVC), polyvinylpyrrolidone; polysiloxanes; polystyrene (PS; celluloses including derivative celluloses such as alkyl celluloses, hydroxyalkyl celluloses, cellulose ethers, cellulose esters, nitro celluloses, hydroxypropylcellulose, and carboxymethylcellulose; polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) (jointly referred to herein as “polyacrylic acids”); polydioxanone and its copolymers; polyhydroxyalkanoates; polypropylene fumarate; polyoxymethylene; poloxamers; poly(butyric acid); trimethylene carbonate; and polyphosphazenes. Examples of preferred natural polymers include proteins such as albumin, collagen, gelatin and prolamines, for example, zein, and polysaccharides such as alginate. Copolymers of the above, such as random, block, or graft copolymers, or blends of the polymers listed above can also be used.

In some embodiments, the nanoparticles have encapsulated therein, dispersed therein, and/or covalently or non-covalently associate with the surface one or more therapeutic or diagnostic agents.

As used herein, the terms “incorporated” and “encapsulated” refers to incorporating, formulating, or otherwise including an active agent into and/or onto a composition that allows for release, such as sustained release, of such agent in the desired application. The terms contemplate any manner by which a therapeutic agent or other material is incorporated into a polymer matrix, including, for example: attached to a monomer of such polymer (by covalent, ionic, or other binding interaction), physical admixture, enveloping the agent in a coating layer of polymer, incorporated into the polymer, distributed throughout the polymeric matrix, appended to the surface of the polymeric matrix (by covalent or other binding interactions), encapsulated inside the polymeric matrix, etc.

In some embodiments, the therapeutic agent can be a small molecule, protein, polysaccharide or saccharide, nucleic acid molecule and/or lipid. Exemplary classes of therapeutic agents include, but are not limited to, analgesics, anti-inflammatory drugs, antipyretics, antidepressants, antiepileptics, antipsychotic agents, neuroprotective agents, anti-proliferatives, such as anti-cancer agent, anti-infectious agents, such as antibacterial agents and antifungal agents, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated. These may be vitamins, supplements such as calcium or biotin, or natural ingredients such as plant extracts or phytohormones.

Exemplary diagnostic agents include paramagnetic molecules, fluorescent compounds, magnetic molecules, and radionuclides. Suitable diagnostic agents include, but are not limited to, x-ray imaging agents and contrast media. Radionuclides also can be used a imaging agents. Examples of other suitable contrast agents include gases or gas emitting compounds, which are radioopaque. Nanoparticles can further include agents useful for determining the location of administered particles. Agents useful for this purpose include fluorescent tags, radionuclides and contrast agents.

Pharmaceutical formulations and methods for the pulmonary delivery of nanoparticles to patients are known in the art. For instance, formulations can be divided into dry powder formulations and liquid formulations. Both dry powder and liquid formulations can be used to form aerosol formulations. The term “aerosol” as used herein refers to any preparation of a fine mist of particles, which can be in solution or a suspension, whether or not it is produced using a propellant.

A further object of the present invention relates to a mucus-penetrating nanoparticle coated with a TG2 inhibitor as described herein.

In some embodiments, the TG2 inhibitor is attached to the surface of the nanoparticle by any conventional method well known in the art, such as described in Hermanson, Greg T. Bioconjugate techniques. Academic press, 2013. In some embodiments, 1-ethyl-3-[3-dimethylaminopropyl] carbodiimide hydrochloride (EDC)-N-hydroxysulfosuccinimide (Sulfo NHS) reactions are used for conjugating the coronaviral polypeptides to the particles. In some embodiments, the particle is conjugated to an avidin moiety that can create an avidin-biotin complex with the biotinylated coronaviral polypeptides and the particles. Additional, appropriate cross-linking agents for use in the invention include a variety of agents that are capable of reacting with a functional group present on a surface of the particle. Reagents capable of such reactivity include homo- and hetero-bifunctional reagents, many of which are known in the art. Heterobifunctional reagents are preferred. A typical bifunctional cross-linking agent is N-succinimidyl(4-iodoacetyl) aminobenzoate (SIAB). However, other crosslinking agents, including, without limitation, dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-maleimidomethyl)cyclohexane-1-carboxylate (SMCC) and 6-hydrazinonicotimide (HYNIC) may also be used. For further examples of cross-linking reagents, see, e.g., S. S. Wong, “Chemistry of Protein Conjugation and Cross-Linking,” CRC Press (1991), and G. T. Hermanson, “Bioconjugate Techniques,” Academic Press (1995).

The invention will be further illustrated by the following figures and examples. However, these examples and figures should not be interpreted in any way as limiting the scope of the present invention.

### FIGURES

FIG. 1: TG2 expression is upregulated in MUC5AC hyperexpressing epithelia A: TG2 and MUC5AC mRNA were quantified by RT-qPCR in in vitro regenerated bronchial epithelia by culturing cells from controls (n=18) and asthma subjects (n=56) at the air-liquid (ALI) interface. Spearman correlation analysis (r) is shown). B: TG2 mRNA upregulation during IL-13-induced mucus cell hyperplasia (black bars) as compared to control epithelial cell differentiation (white bars) in ALI culture. n=3.

FIG. 2: TG2 upregulation is observed in in vitro regenerated bronchial epithelia in asthma and is associated with poorer mucociliary clearance A: TG2 mRNA were quantified by RT-qPCR in ALI-regenerated bronchial epithelia from controls (n=18) and asthma subjects (n=56). B: Mean velocity of fluorescent microspheres deposited on ALI-epithelia from controls and TG2-low or TG2-high asthma patients and analysed using a Zeiss videomicroscope and ImageJ (MTrack3 Plugin). Median values are shown. *p<0.05

FIG. 3: TG2 expression levels are upregulated in ALI-regenerated epithelia from cystic fibrosis and asthma patients and correlate with TGF-beta2 expression levels. TG2 and TGF-beta2 mRNA levels were quantified by microarray (A) or RT-qPCR (B and C) in cystic fibrosis (A and B, n=4) and asthma (C, n=49) patients. A: Data are expressed as mean S.E.M. * p<0.05. B and C: Correlation analysis of TG2 and TGF-beta2 levels (Spearman (r)) in cystic fibrosis (B) and asthma (C) sets.

FIG. 4: Respiratory mucins are TG2 substrates Respiratory mucins secreted by ALI-regenerated epithelia were reduced, alkylated and subsequently exposed to human recombinant TG2 with a biotinylated amine (BAP) in the absence (line 1) or presence of ZDON (line 3) or of its vehicle (line 2) for 24h @ 37° C. Mucins were then separated using hybrid SDS agarose/polyacrylamide gel, transferred on a PVDF membrane and BAP incorporation was detected using streptavidine-HRP.

FIG. 5: The TG2 inhibitor ZDON decreases MUC5AC tethering to the apical surface of hypersecretory epithelia MUC5AC was detected by immunofluorescence in ALI-regenerated hypersecretory epithelia after extensive apical washes with PBS in sections (A and B) or in whole-mount preparations (C and D). Whole-mount images represent stacks from optical sections done through the mucus (parallel to the plane of the epithelium). epi, epithelium. Extracellular mucus was quantified after beta-tubulin staining to delineate cell apex, and expressed as sum intensities (in nm 2 or nm 3) (n=2).

FIG. 6: The TG2 inhibitor ZDON tends to ameliorate mucociliary clearance in hypersecretory epithelia A: Paths of fluorescent microspheres deposited on IL-13-stimulated hypersecretory epithelia. The final image from a 30-second image sequence (every 5 ms) is shown (ImageJ, Z Project). B: Quantification of travelled distance (ImageJ, Plugin MTrack3). Median values are shown. n=2.

### EXAMPLE

Methods: The bronchial epithelium from asthmatic patients, cystic fibrosis or control subjects was reconstituted in vitro by culturing cells at the air-liquid interface. Hyper secretory differentiation was modeled by exposing control bronchial epithelial to IL-13. Expression of TG2 and MUC5AC was assessed by RT-qPCR and immunohistochemistry/immunofluorescence. Mucin modification by TG2 was evaluated by BAP incorporation and Western-blot analysis. Mucus tethering to the apical surface was studied by detecting MUC5AC after extensive apical washes with PBS. The role of TG2 was evaluated using the pharmacological inhibitor ZDON. Mucociliary clearance was evaluated by video microscopy using fluorescent microspheres.

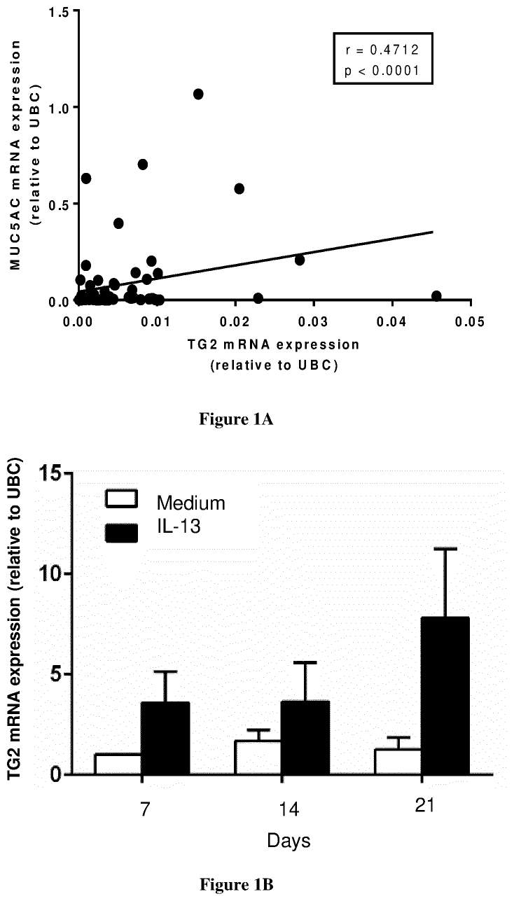

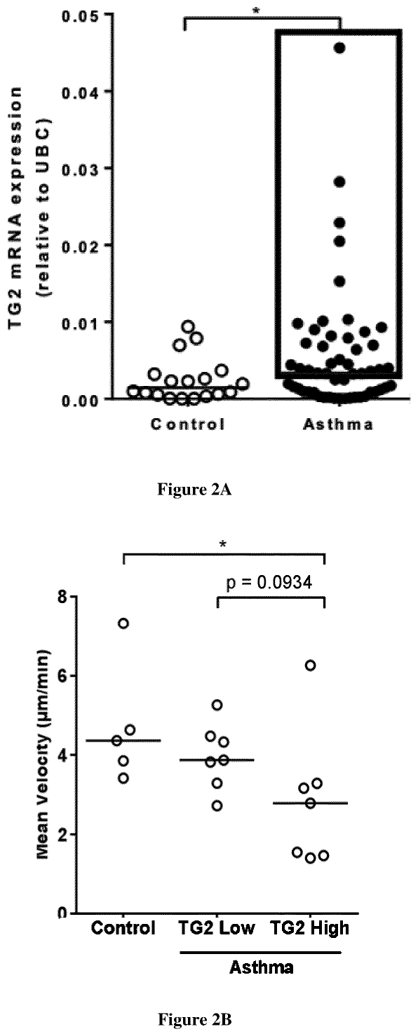

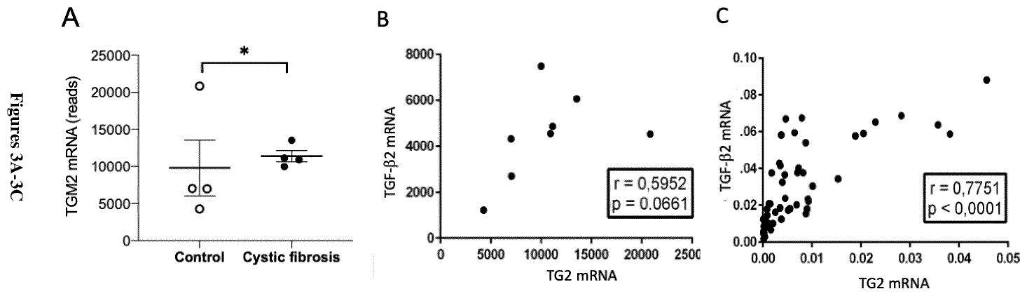

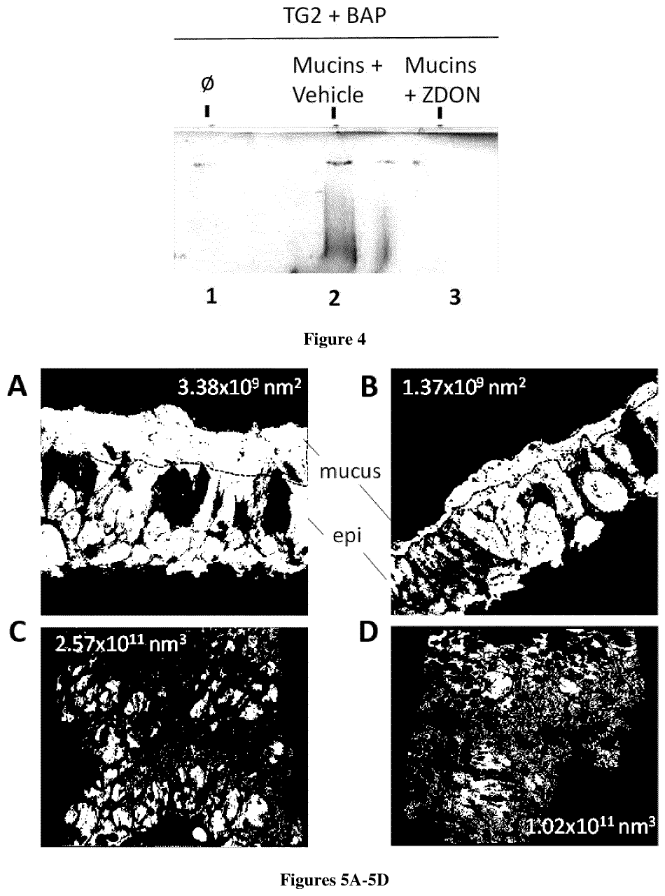

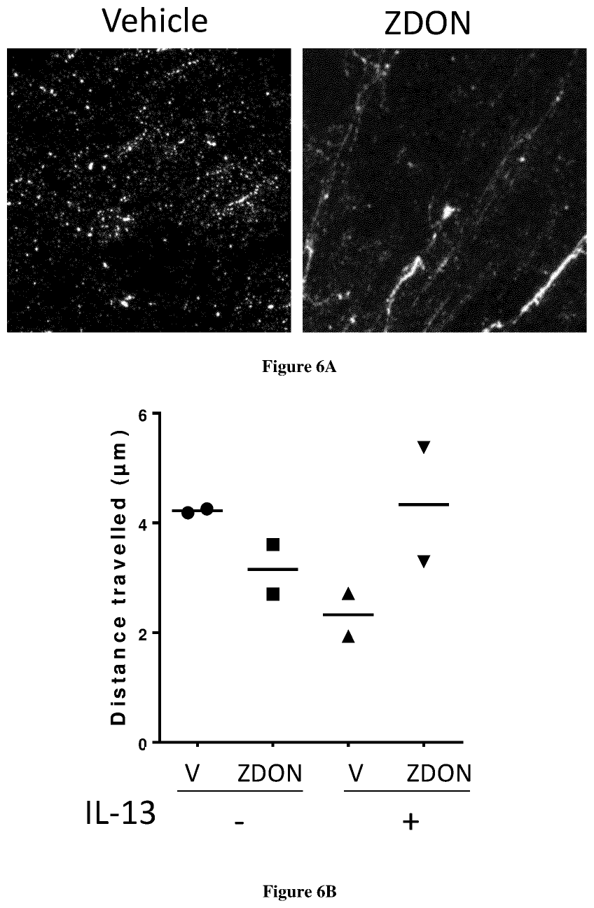

Results: TG2 expression was upregulated in a sub-group of asthma patients and upon IL-13-mediated hypersecretory differentiation and correlated with MUC5AC expression (FIGS. 1A-1B). TG2-high epithelia showed decreased microsphere transport. Indeed, TG2 upregulation is observed in in vitro regenerated bronchial epithelia in asthma and is associated with poorer mucociliary clearance (FIGS. 2A-2B). TG2 colocalized with MUC5AC at the surface of the bronchial epithelium in asthma. TG2 expression levels are upregulated in ALI-regenerated epithelia from cystic fibrosis and asthma patients and correlate with TGF-beta2 expression levels (FIGS. 3A-3C). TG2-exposed respiratory mucins incorporated the biotinylated amine BAP (FIG. 4). IL-13 promoted MU5AC tethering to in vitro reconstituted hypersecretory epithelium (FIGS. 5A-5D), and this was blocked by ZDON. Indeed, the TG2 inhibitor ZDON tends to ameliorate mucociliary clearance in hypersecretory epithelia (FIGS. 6A-6B).

Conclusion: TG2 would participate in respiratory mucin modifications in asthma, and contribute to mucus tethering to the airway wall.

### REFERENCES

Throughout this application, various references describe the state of the art to which this invention pertains. The disclosures of these references are hereby incorporated by reference into the present disclosure.

1. A method of improving mucociliary clearance, increasing the liquefaction of mucus, preventing mucus tethering to the airway wall, preventing extensive airway mucus plugging and/or preventing mucus occlusion of an airway lumen in a patient suffering from a chronic airway disease, comprising administering to the patient a therapeutically effective amount of a TG2 inhibitor.

2. (canceled)

3. (canceled)

4. (canceled)

5. (canceled)

6. The method according to claim 1, wherein the patient has a chronic airway disease which causes abnormal or excessive viscoelasticity or cohesiveness of mucus.

7. The method according to claim 1, wherein the patient has a chronic airway disease selected from, cystic fibrosis (CF), chronic obstructive pulmonary disease, bronchiectasis and asthma.

8. The method according to claim 1, wherein the patient suffers from asthma.

9. The method according to claim 1, wherein the TG2 inhibitor is a small organic molecule or an antibody.

10. The method according to claim 1, wherein the TG2 inhibitor is an inhibitor of gene expression that is a siRNA, an antisense oligonucleotide or a ribozyme.

11. A method for increasing the lung delivery of nanoparticles in a patient in need thereof comprising administering the nanoparticles in combination with an amount of a TG2 inhibitor.

12. The method of claim 11 wherein the nanoparticles have encapsulated therein, dispersed therein, and/or covalently or non-covalently associated with a surface thereof one or more therapeutic or diagnostic agents.

13. The method of claim 12 wherein the one or more therapeutic agents is selected from the group consisting of analgesics, anti-inflammatory drugs, antipyretics, antidepressants, antiepileptics, antipsychotic agents, neuroprotective agents, anti-proliferatives, such as anti-cancer agent, anti-infectious agents, such as antibacterial agents and antifungal agents, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics.

14. The method of claim 12 wherein the one or more diagnostic agents is selected from the group consisting of paramagnetic molecules, fluorescent compounds, magnetic molecules, and radionuclides.

15. A mucus-penetrating nanoparticle coated with a TG2 inhibitor.

16. The method according to claim 8, wherein the asthma is severe asthma.
